(19) United States
(12) Patent Application Publication
(10) Pub. No.: US 2018/0009036 A1
(43) Pub. Date: Jan. 11, 2018

(54) METHODS OF SYNTHESIZING DENDRITIC GOLD NANOPARTICLES

(71) Applicant: The Board of Regents of the University of Oklahoma, Norman, OK (US)

(72) Inventors: Chuanbin Mao, Norman, OK (US); Penghe Qiu, Norman, OK (US)

(21) Appl. No.: 15/635,948

(22) Filed: Jun. 28, 2017

Related U.S. Application Data
(60) Provisional application No. 62/358,929, filed on Jul. 6, 2016.

(51) Int. Cl. B22F 9/24; A61K 41/00; B22F 1/00

(52) U.S. Cl. CPC B22F 9/24; B22F 1/0044; A61K 41/0052; B22F 2301/255; B22F 2009/245

(57) ABSTRACT

Methods of synthesizing gold nanodendrites (AuNDs) using amines, such as long chain amines, as a structural directing agent are disclosed. Degree of branching (DB) of the AuNDs can be tuned by adjusting certain synthetic parameters, such as solvent type, and the type and concentration of the long chain amines. DB control results in dramatic tunability of the optical properties of the AuNDs in the near infrared (NIR) range enabling improved performance, for example as a photothermal cancer therapeutic.

### CROSS-REFERENCE TO RELATED APPLICATIONS

This application claims benefit under 35 U.S.C. 119(e) of U.S. Provisional Application Ser. No. 62/358,929, filed Jul. 6, 2016, which is hereby expressly incorporated herein by reference in its entirety.

### STATEMENT REGARDING FEDERALLY SPONSORED RESEARCH OR DEVELOPMENT

Not applicable.

### BACKGROUND

Solar energy is the most convenient, most abundant and cost-free energy source on earth. Many different types of materials, such as semiconductors, carbon-based black materials and plasmonic gold nanoparticles, have been developed for solar light capture. Among them, plasmonic gold nanoparticles are one of the most important solar absorbers due to their extremely high molar extinction coefficient, highly tunable optical properties and the generation of surface free electrons (surface plasmon) upon light irradiation. Solar irradiation contains continuous light ranging from UV to infrared region (300-2500 nm), however, the intensity at any individual wavelength is very low. Therefore, in order to effectively harness solar irradiation, a solar light absorber should have broad band optical properties.

Gold nanoparticles have been used to absorb certain wavelengths of solar light. Compared to gold nanoparticles of other shapes, such as nanospheres, nanorods, nanocubes and nanostars, dendritic gold nanoparticles (also referred to herein as “gold nanodendrites” or “AuNDs”) tend to have the widest optical bands that would allow the most effective solar energy absorption.

Nanodendrites (NDs) are a group of nanoparticles that have the characteristic hyperbranched nanostructures. Owing to their extremely large surface area that can greatly enhance the catalytic, electrochemical and drug delivery performance, NDs have recently attracted intensive research attention. Previously, NDs of metals and bimetals, including Pt, Pd, Au, Au—Pd, Au—Pt, Pd—Pt, Pd—Co, Pd—Ni, and Pt—Cu, have been synthesized by a number of different approaches. In polymer science, the degree of branching (DB) is an important parameter that determines the chemical, physical and mechanical properties of dendritic polymers. Similarly, for inorganic dendritic nanoparticles (NPs), many of their properties such as optical extinction and catalysis might also be dependent on the DB. Although NDs with different DB can be found in particles prepared by different methods, there has not been a single synthetic system that can fabricate NDs with tunable DB. For this reason, it has remained unknown how the DB would affect the various properties of dendritic NPs.

Conventional synthesis of gold nanodendrites (AuNDs) has been achieved by using polymers such as polypyrrole and polyaniline, and the surfactant, bis(amidoethyl-carbamoylethyl)octadecylamine in aqueous phase. However, the optical properties of AuNDs prepared by these methods, although exhibiting certain levels of broadband absorption, are relatively weak in terms of tunability. And furthermore, none of the conventional methods can generate nanoparticles that absorb over the whole light range of solar light (300 nm-2500 nm). In this sense, the full efficiency of solar energy capture by these gold nanodendrites is yet to be achieved. Thus, synthetic methods that allow more controllable tuning of optical properties of gold nanodendrites are still desired.

Further, as a non-invasive approach for cancer therapy, photothermal treatment has attracted much attention in the past decade. Due to the limited penetration depth of near infrared light into tissues, photothermal therapy has been considered more suitable for breast cancers than other types of deep cancers. Ideal photothermal probes should have high molar extinction coefficient, so that they can absorb light more efficiently to generate more heat.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness.

FIG. 1 shows transmission electron microscopy (TEM) images of gold nanoparticles (AuNPs) synthesized in ethanolic solutions of amines of different carbon chain lengths: (a) butylamine, (b) octylamine, (c) dodecylamine, (d) hexadecylamine, (e) octadecylamine and (f) oleylamine. Dendritic structures can be seen unambiguously in amines with longer chains (c-f). Scale bar: 50 nm, applies to all images.

FIG. 2 shows TEM images demonstrating control over gold nanodendrite (AuND) size by adjusting stoichiometry of gold seeds and HAuCl₄. In the figure, concentration of HAuCl₄ was kept constant. The amount of gold seeds used was 16, 4, 2 and 0.5×10¹¹/ml (a-d) respectively. Scale bar: 40 nm, applies to all images.

FIG. 3 shows TEM images of AuNDs of high (a), medium (b), and low (c) degree of branching for the photothermal studies. The AuNDs were synthesized in (a) 0.1 M of hexadecylamine in ethanol, (b) 0.4 M of oleylamine in chloroform and (c) 1.0 M of oleylamine in chloroform, under the same amount of gold seeds and HAuCl₄. The average diameter of branches is 7.0±0.5 nm, 6.7±0.2 nm, and 6.7±0.4 nm in a, b and c respectively. Scale bar: 100 nm, applies to all images.

FIG. 4 graphically demonstrates dependence of optical and photothermal properties of AuNDs on degree of branching (DB), (a) Extinction spectra; (b) Molar extinction coefficient of AuNDs at 808, 980 and 1064 nm; (c) Temperature profiles of aqueous AuNDs solutions irradiated by 808 and 980 nm laser, both with a power density of 1.0 W/cm², for 5 min; (d) In vitro study of cell viability after irradiation of 808 and 980 nm laser of different power densities. H, M, and L correspond to AuNDs of high, medium and low DB respectively. H-100, H-50 and H-25 in c mean 100, 50 and 25 μg/ml AuNDs of high DB, respectively, and so as other symbols with the prefix M and L.

FIG. 5 shows results of in vivo photothennal treatment of MCF-7 tumors by using AuNDs of different DB. (a, b), Posttreatment tracking of tumor volume in groups treated by 808 nm and 980 nm laser, respectively; (c) Photographs of a typical mouse in each group on day 1 and day 12 posttreatment; (d) Real-time tracking of temperature increase inside tumors during the photothermal treatment by an infrared camera.

FIG. 6 shows TEM images of AuNPs prepared by using primary (dodecylamine, a), secondary (N-methyldodecylamine, b), tertiary (N,N-dimethyldodecylamine, c) and quaternary (dodecyltrimethylammonium bromide, d) amines as structure-directing agents. Dendritic structures can only be induced by the primary amine. Scale bar: 50 nm.

FIG. 7 is a TEM image showing AuNPs synthesized in 0.1 M of lauric acid in ethanol.

FIG. 8 shows low magnification TEM images of AuNDs of controlled size, corresponding to high magnification TEM images of the AuNDs shown in FIG. 2.

FIG. 9 shows TEM images of formation of dendritic structures on gold seeds of different shapes: (a-c) triangular nanoplates; and (d-i) long nanorods. The degree of overgrowth on each type of seed was controlled by using the same amount of seeds, but one (a, d), two (b, e) and six (c, f) times the volume of HAuCl₄, respectively. Images d, e & f are magnified segments of the nanorods shown in g, h & I, respectively. Arrow in c indicates the edge of the original seed. Scale bars: 40 nm in a-f and 200 nm in g-i.

FIG. 10 shows the morphological evolution of AuNDs over the reaction time. TEM images of two typical nanoparticles obtained after reaction for 2 (a, b), 3 (c, d), 4 (e, f), 7 (g, h) and 30 min (i, j) are presented in this figure. All scale bars are 20 nm.

FIG. 11 shows high resolution TEM images of a simply branched nanoparticle. The results indicate that the as-synthesized AuNDs are polycrystalline. The overall image of the nanoparticle is shown in the inset of a. HRTEM images of a tip and a branch junction, as framed in inset a, are shown in a & b, respectively. Scale bar in inset a: 10 nm.

FIG. 12 is a schematic representation of a possible formation mechanism of AuNDs of the methods of the present disclosure. Step 1-2, Self-assembly of Au³⁺ and long chain amines into rod-like nanostructures; Step 2-3, Ascorbic acid alone cannot induce the growth of NPs; Step 2-4, In the presence of seeds as catalysts, reaction can be initiated once the rod-like nanostructures are in contact with the seeds; Step 4-6, Due to the confinement of Au³⁺ inside the rod-like nanostructures, deposition of gold atoms can only take place anisotropically along the rods, which resulted in the first-generation branches; Step 6-8, first generation branches can serve as new catalytic sites immediately for the deposition of second-generation branches; Step 8-9, the growing process is repeated in such a manner to eventually form a multiple-generation branched structure.

FIG. 13 depicts a mechanism study on the formation of AuNDs by Dynamic Light Scattering (DLS) and TEM. (a) DLS measurement of size distribution of particles formed upon the addition of HAuCl₄ into the amine solutions of different carbon chain lengths (C4-butylamine, C8-octylamine, C12-dodecylamine, C16-hexadecylamine, OAm-oleylamine). (b-d) Low- and high-magnification TEM images of typical rod-like nanostructures formed by HAuCl₄ and long chain amines. Tiny gold nanoparticles can only be observed inside the rod-like profile, suggesting that gold ions have been confined in the nanostructure and cannot diffuse freely in solution. Scale bars: 200 nm, b; 100 nm, c; 50 nm, d.

FIG. 14 depicts evidence of the possible bundling of the rod-like structures during the treatment for TEM study. Gold nanoparticles arrays inside the dark lines are likely formed from the individual rod-like structures; while those inside the red lines are of the bundled rod-like structures.

FIG. 15 depicts TEM images showing the effect of solvent type on the DB of the AuNDs. AuNDs synthesized in 0.1 M of hexadecylamine/ethanol solutions (a, c) and in 0.1 M of hexadecylamine/chloroform solutions (b, d). Concentration of seeds used: 10×10¹¹/ml (a, b) and 2.5×10¹¹/ml (c, d). Scale bar: 50 nm.

FIG. 16 show UV-Vis-NIR spectra showing the optical extinction of AuNDs prepared in 0.1 M of hexadecylamine/chloroform solutions. Amount of seeds used is 10, 2.5, 0.5 and 0.1×10¹¹/ml in a-d respectively.

FIG. 17 depict TEM images showing the effect of amine concentration on DB of AuNDs synthesized in oleylamine/chloroform solutions. Concentration of oleylamine was 0.1, 0.4, 0.7 and 1.0 M from a-d, respectively. All other parameters, like the amount of seeds and HAuCl₄, used for the synthesis were kept constant. Scale bar: 50 nm.

### DETAILED DESCRIPTION

Prior to the presently disclosed work, adequate control over the degree of branching (DB) of dendritic nanoparticles has been impossible. The present disclosure describes methods of synthesizing gold nanodendrites (AuNDs) using long chain amines as a structural directing agent. We have discovered that the DB can be tuned facilely by adjusting certain synthetic parameters, such as solvent type, and the type and concentration of the long chain amines. DB tuning results in dramatic tunability in the optical properties in the near infrared (NIR) range enabling improved performance as a photothermal cancer therapeutic. The resultant AuNDs have significantly higher molar extinction coefficient than that of other types of gold nanoparticles, such as nanospheres, nanorods, and nanocages, thus they can act as better probes for photothermal cancer therapy. As described below, in vitro and in vivo studies demonstrated that AuNDs with a higher DB are more efficient in photothermal tumor destruction under a lower wavelength NIR irradiation, while AuNDs with a lower DB performed better in tumor destruction under a higher wavelength NIR irradiation. Thus, the tunable optical properties of AuNDs, such as absorbability in the NIR range, enable the selective determination of the particular laser wavelengths for the best cancer therapeutic performance or other uses to which the AuNDs described herein may be put.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 300 nm to 2500 nm therefore refers to and includes all values or ranges of values, and fractions of the values and integers within said range, including for example, but not limited to 400 nm to 2250 nm, 400 nm to 2000 nm, 600 nm to 2250 nm, 600 nm to 2000 nm, 400 nm to 1750 nm, 750 nm to 2000 nm, 750 nm to 1750 nm, 750 nm to 1600 nm, 400 nm to 1600 nm, and 800 nm to 1200 nm. Any two values within the range of 300 nm to 2500 nm therefore can be used to set the lower and upper boundaries of a range in accordance with the embodiments of the present disclosure.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The ten is “subject” and “patient” are used interchangeably herein and will be understood to refer to a warm blooded animal, particularly a mammal or bird. Non-limiting examples of animals within the scope and meaning of this term include dogs, cats, rats, mice, guinea pigs, horses, goats, cattle, sheep, zoo animals, Old and New World monkeys, non-human primates, and humans.

“Treatment” refers to therapeutic treatments. “Prevention” refers to prophylactic treatment measures to stop a condition from occurring. The term “treating” refers to administering the composition to a patient for therapeutic purposes, and may result in an amelioration of the condition or disease.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

The novel methods of the present disclosure employ a completely different formula (e.g., one or more types of long chain primary amines in ethanol or chloroform solution) than has been previously used to prepare dendritic gold nanoparticles (polypyrrole and/or polyaniline and a non-commercial surfactant, bis(amidoethyl-carbamoylethyl)octadecylamine in aqueous phase). The uniformity of AuNDs produced has been significantly improved, for example the size manipulation has been remarkably simplified. Further, unlike in conventional methods, the DB of the dendritic nanostructures can be controlled. The success in manipulating the DB enables the selective tuning of the optical absorption properties of gold nanodendrites within the solar spectrum. As a result, we can make gold nanodendrites that can absorb wavelengths in the visible, near infrared, or the whole spectrum of the solar light. Such an optical tunability has important implications for solar-based applications, and is is not achievable by conventional methods. Further, the surface chemistry of the presently disclosed AuNDs can be tuned facilely, whereas gold nanodendrites prepared by the conventional polypyrrole and polyaniline methods are buried inside a thick polymer layer, thus limiting the modifications which can be made in their surface chemistry and significantly limiting their applications. Moreover, while there are currently many types of nanoparticles available on the market for different applications, there are no commercially available AuNDs.

In certain non-limiting embodiments, the AuNDs of the present disclosure can be used in photothermal therapy of cancers, such as those using a monochromic infrared laser. The gold nanodendrites of the present disclosure will be the first nanodendrites that employ sunlight as the irradiation source for photothermal therapy of cancers. The gold nanodendrites of the present disclosure can also be used as carriers of therapeutic compounds and genetic materials. The AuNDs of the present disclosure can also be used for other solar-based applications, such as solar-based thermoelectric generation, photocatalysis, and photochemical reactions. Furthermore, since the AuNDs of the present disclosure possess extremely larger surface area, they can also be used as drug and gene carriers.

Certain novel embodiments of the present disclosure, having now been generally described, will be more readily understood by reference to the following examples, which are included merely for purposes of illustration of certain aspects and embodiments of the present disclosure, and are not intended to be limiting. The following detailed examples are to be construed, as noted above, only as illustrative, and not as limiting of the present disclosure in any way whatsoever. Those skilled in the art will promptly recognize appropriate variations from the various compositions, structures, components, procedures and methods.

Methodology

In at least certain embodiments, the AuNDs of the present disclosure can be manufactured using procedures described in the following non-limiting examples.

### EXAMPLE 1

Materials

Gold chloride (HAuCl₄, 99.9%) was purchased from Strem Chemicals. Thiolated polyethyleneglycol (HS-PEG, 2k) was bought from Laysan Bio. Ascorbic acid (98%), Sodium citrate tribasic dehydrate (99%), Sodium borohydride (NaBH₄, 98%), Oleylamine (70%, technical grade) and Polyvinylpyrrolidone (PVP, MW 10,000) were purchased from Sigma-Aldrich. Hexadecylamine (90%), were purchased from Fluka. Ethanol and Tetrahydrofuran (THF) were all of analytical grade and purchased from Fisher. All chemicals were used as received.

Synthesis and phase transfer of AuNDs

Synthesis of Spherical Gold Nanoparticle (AuNP) Seeds

Seed gold nanoparticles (NPs) were synthesized through the reduction of gold chloride (HAuCl₄) by sodium citrate. A solution containing 700 μl of 60 mM HAuCl₄ and 45 ml of deionized water was first boiled for 5 min, followed by addition of 5 ml of 38.8 mM sodium citrate. The mixture was allowed to react for about 15 min, and then cooled to room temperature. Prior to use of the as-prepared AuNPs as seeds for the AuND synthesis, they were coated with a layer of PVP. The PVP coating was done by simply dissolving 0.2 g of the polymer into the AuNP solution and stirring for 24 h. The PVP-stabilized AuNPs were then centrifuged and re-dispersed into 10 ml of ethanol. Afterward, they can be readily used for the AuND synthesis.

Synthesis of AuNDs

To perform a synthesis, 4 ml of 0.1 M ethanolic solution of primary amines (e.g., hexadecylamine or oleylamine), 20 μl of 60 mM HAuCl₄ in ethanol, varying amount of gold seeds, and 15 μl of 0.4 M ascorbic acid in methanol were added subsequently into the reaction vial. The solutions were then mixed vigorously by hand for a few seconds, and then left on a rocker shaker for about 30 min, during which AuNDs form. The nanoparticles were then centrifuged, washed once with chloroform and ethanol and finally redispersed into 2 ml of THF. The degree of branching was controlled by changing the solvent and concentration of amines in the synthetic system.

Phase Transfer of the Hydrophobic AuNDs into Water

To 2 ml of the THF dispersion of AuNDs, 2 mg of HS-PEG was added. The resulting solution was gently sonicated and left on the bench for overnight. Afterwards, the AuNDs were collected by centrifugation, washed once with ethanol, and redispersed into water.

Measuring Optical Properties of AuNDs

The optical properties of AuNDs were measured on a regular UV-vis-NIR spectrometer.

### EXAMPLE 2

Materials and reagents

Gold chloride (HAuCl₄, 99.9%) was purchased from Strem Chemicals. Thiolated polyethyleneglycol (HS-PEG, 2k) was bought from Laysan Bio. Ascorbic acid (98%), Sodium citrate tribasic dehydrate (99%), Sodium borohydride (NaBH₄, 98%), Butylamine (99.5%), Octylamine (99%), Dodecylamine (98%), Oleylamine (70%, technical grade) Polyvinylpyrrolidone (PVP, MW 10,000), and Cetyl trimethyl ammonium bromide (CTAB, 99%) were purchased from Sigma-Aldrich. Hexadecylamine (90%), Octadecylamine (90%), and 4-Nitrophenol (99.5%) were purchased from Fluka. Ethanol, Tetrahydrofuran (THF) and Dimethylformamide (DMF) were all of analytical grade and purchased from Fisher. All chemicals were used as received.

Synthesis and phase ransfer of AuNDs

Synthesis of Spherical AuNPs Seeds.

Seed gold nanoparticles were synthesized through the reduction of gold chloride (HAuCl₄) by sodium citrate. The solution containing 700 μl of 60 mM HAuCl₄ and 45 ml of deionized water was first boiled for 5 min, followed by addition of 5 ml of 38.8 mM sodium citrate. The mixture was allowed to react for about 15 min, and then cooled to room temperature. Before the as-prepared AuNPs were used as seeds for the AuNDs synthesis, they were coated with a layer of PVP. The PVP coating was done by dissolving 0.2 g of the polymer into the AuNPs solution and stirring for 24 h. The PVP stabilized AuNPs were then centrifuged and re-dispersed into 10 ml of ethanol. Afterwards, they could be readily used for the AuNDs synthesis.

Synthesis of AuNDs.

To perform a synthesis, 4 ml of 0.1 M ethanolic solutions of several amines (butylamine, octylamine, dodecylamine, hexadecylamine, octadecylamine, oleylamine, N-methyldodecylamine, N,N-dimethyldodecylamine and dodecyltrimethylammonium bromide), 20 of 60 mM HAuCl₄ in ethanol, varying amount of seeds, and 15 μl of 0.4 M ascorbic acid in methanol were added subsequently into the reaction vial. The solutions were then mixed vigorously by hand for a few seconds, and then left on a rocker shaker for about 30 min, during which AuNDs formed. The nanoparticles were then centrifuged, washed once with chloroform and ethanol and finally redispersed into 2 ml of THF. A similar set of reactions was carried out using amine solutions of chloroform instead of ethanol.

Phase Transfer of the Hydrophobic AuNDs into Water.

To 2 ml of THF dispersion of AuNDs, 2 mg of HS-PEG was added. The solution was gently sonicated and left on the bench for overnight. Afterwards, the nanoparticles were collected by centrifugation, washed once with ethanol and redispersed into water.

Photothermal Study of AuNDs

Measuring temperature profile in the aqueous solutions of AuNDs.

The AuNDs of different DB for the photothermal study were all prepared with the same amount of seeds and HAuCl₄, but with different concentrations of the long chain amines. To measure the temperature change, aqueous solutions of AuNDs (100 μl various concentrations, i.e. 25, 50 and 100 μg/ml) were irradiated by 808 or 980 nm laser (1.0 W/cm²) for 5 min in a 96-well plate. Temperature of the solution was collected every 30 seconds during the irradiation by using an infrared camera (ICI 7320P).

In Vitro Photothermal Therapy

The MCF-7 cells used for this study were obtained from ATCC, and cultured in Eagle's Minimal Essential Medium (EMEM, ATCC) with 10% fetal bovine serum (Gibco, Inc). MCF-7 cells were seeded on a 24-well culture plate for 12 h. Then the AuNDs, prepared with 20 μl seeds, were added at a final concentration of 100 μg/ml and co-cultured with the cells for another 12 h. After that, free nanoparticles were removed by gentle wash. The laser (808 and 980 nm) treatment was started at an initial power density of 2.0 W/cm² and a spot size of 5 mm for 5 min. The cells were cultured for another 4 h after the laser treatment. Thereafter, cells were stained with the LIVE/DEAD® Fixable Dead Cell Stain Kits (Invitrogen, USA). In this assay, the live and dead cells showed red and green colors under fluorescence microscope, respectively. Cell viability was obtained by counting about 300 cells in each sample. For each type of AuNDs, 3 samples were used for the study.

In Vivo Photothermal Therapy.

The nude mice used for in vivo photothermal therapy were purchased from Harlan Lab (Athymic Nude-Foxn1^{nu}, 3-5 weeks, female). To build tumor-bearing mice, 2.5×10⁶ −3×10⁶ cells with 0.1 ml of saline were injected hypodermically on the left flank of each mice. In vivo experiments were carried out when the tumors reached an average diameter of around 5 mm. 200 μl of PEG coated AuNDs at 5.0 mg/ml in PBS were injected through tail vein of each mouse. Twenty four hours after the injection, tumors were irradiated by 808 or 980 nm laser at 1.0 W/cm² for 5 min. A total of 5 mice were used for each group. The tumor size was recorded every 3 days after the laser treatment.

Results and discussion

Synthesis, Size Manipulation and Mechanism of Formation of AuNDs

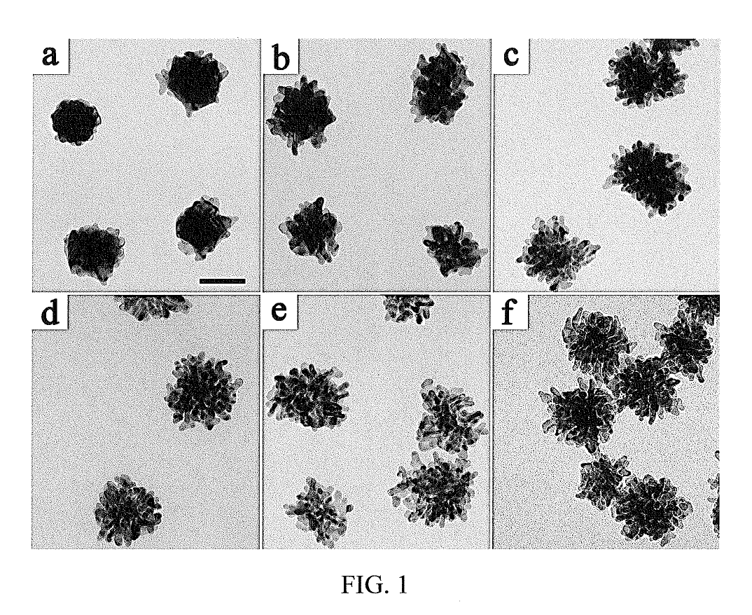

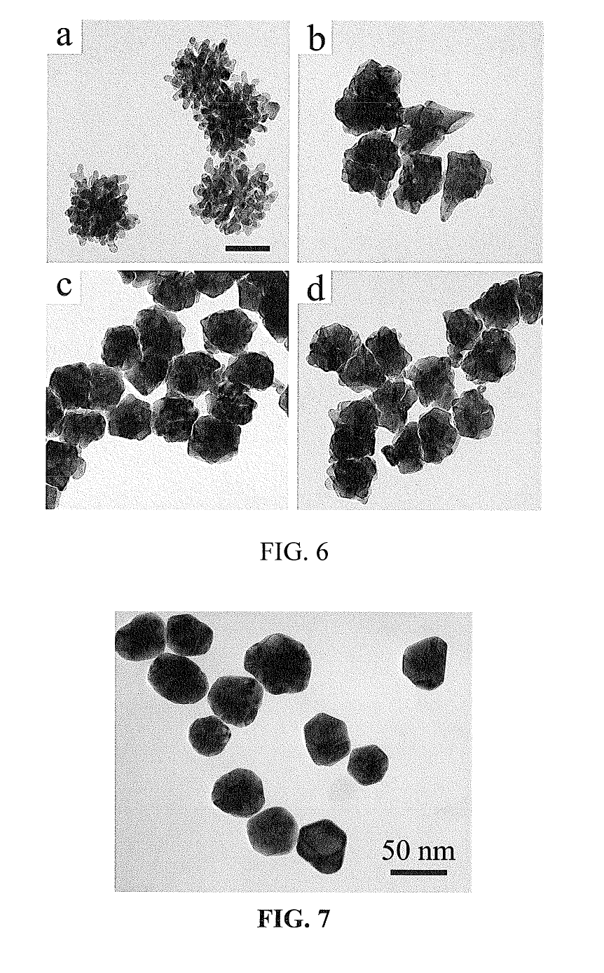

Synthesis of gold NPs (AuNPs) was carried out through a seed-mediated reduction of HAuCl₄ by ascorbic acid in ethanol. Amines of different carbon chain lengths were then introduced as structural-directing agents. AuNPs obtained from 6 different amines are shown in FIG. 1. An apparent morphology dependence of AuNPs on the carbon chain length of the amines can be noticed immediately. Short chain amines, i.e. butylamine and octylamine, can only produce NPs of a few branches (FIGS. 1a-b); while hyperbranched or dendritic structures are seen unambiguously on all of the NPs that were prepared with long chain amines, including dodecylamine, hexadecylamine, octadecylamine and oleylamine (FIGS. 1 c-f). The seed-mediated synthesis was also carried out using secondary, tertiary and quaternary amines with long carbon chains, including N-methyldodecylamine, N,N-dimethyldodecylamine and dodecyltrimethylammonium bromide in comparison to using dodecylamine, however, no hyperbranched structures were produced using the secondary, tertiary and quaternary amines (FIG. 6). Further, long chain fatty acids (non-aminated), such as lauric acid, only results in solid nanoparticles with smooth surface (FIG. 7). Thus, according to the above observation, it is reasonable to conclude that controlled formation of AuNDs is feasible only using long chain primary amines.

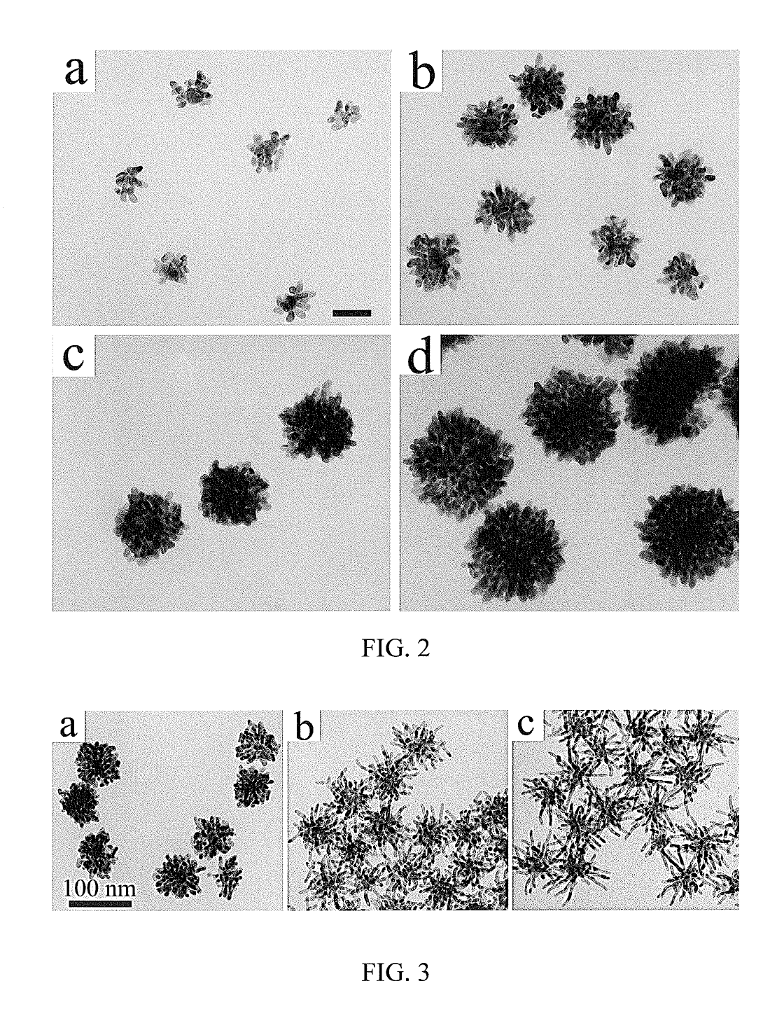

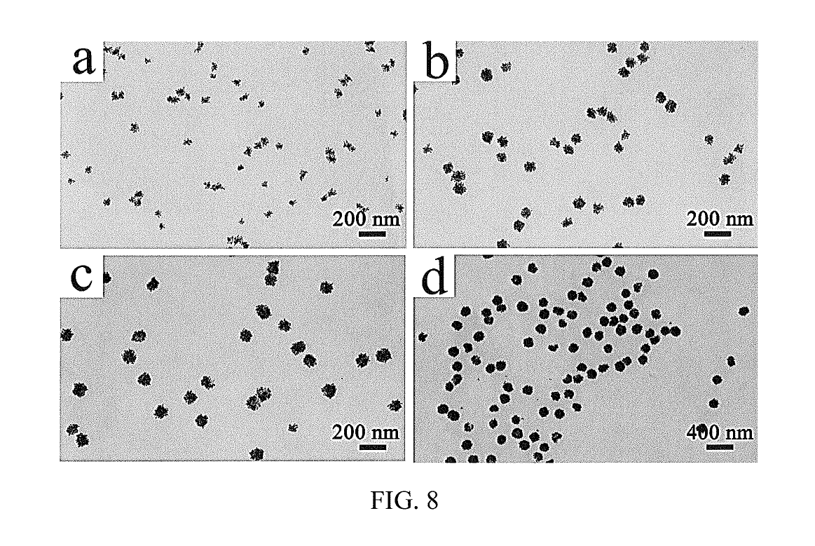

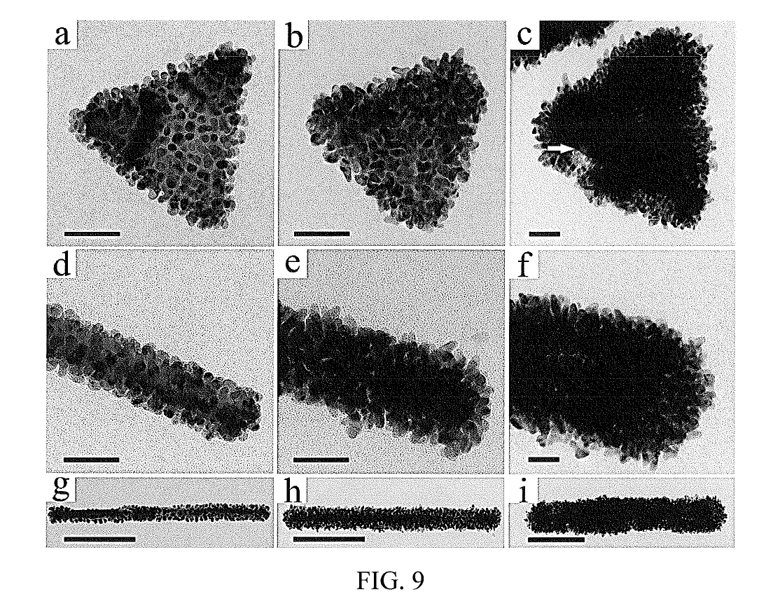

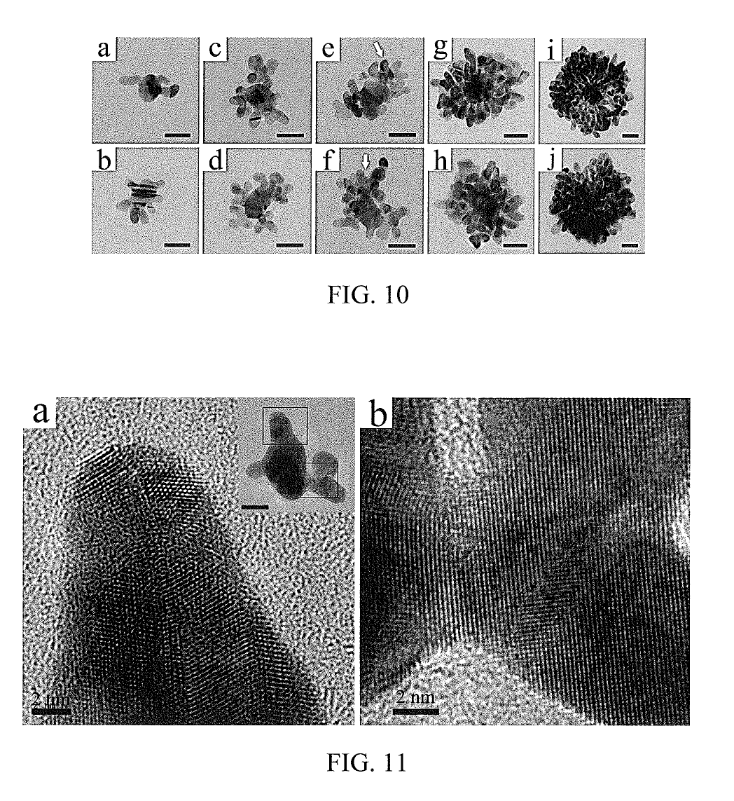

In the synthetic methods of the present disclosure, size manipulation of the AuNDs can be achieved by adjusting the stoichiometry of the precursors. The TEM images in FIG. 2 and FIG. 8 show AuNDs prepared by using different amounts of seeds while keeping all other reagents constant. From these images, it is evident that the size of AuND can be changed according to the amount of seeds. Larger AuNDs are obtained by using fewer seeds and smaller AuNDs are obtained using a larger number of seeds. Alternatively, instead of changing the amount of seeds, size tuning can also be achieved by reducing the quantity of HAuCl₄. The dendritic structures can be grown onto gold seeds of arbitrary shapes. For example, when triangular gold nanoplates and long gold nanorods were used as seeds, the branches could still grow in a well-controlled manner (FIG. 9). The investigation into the morphological evolution of AuNDs over reaction time showed that the overall dendritic structure was produced through stepwise growth of branches (FIG. 10). In addition, high-resolution TEM (HRTEM) examination of AuNDs showed that the as-synthesized AuNDs are of polycrystalline nature (FIG. 11).

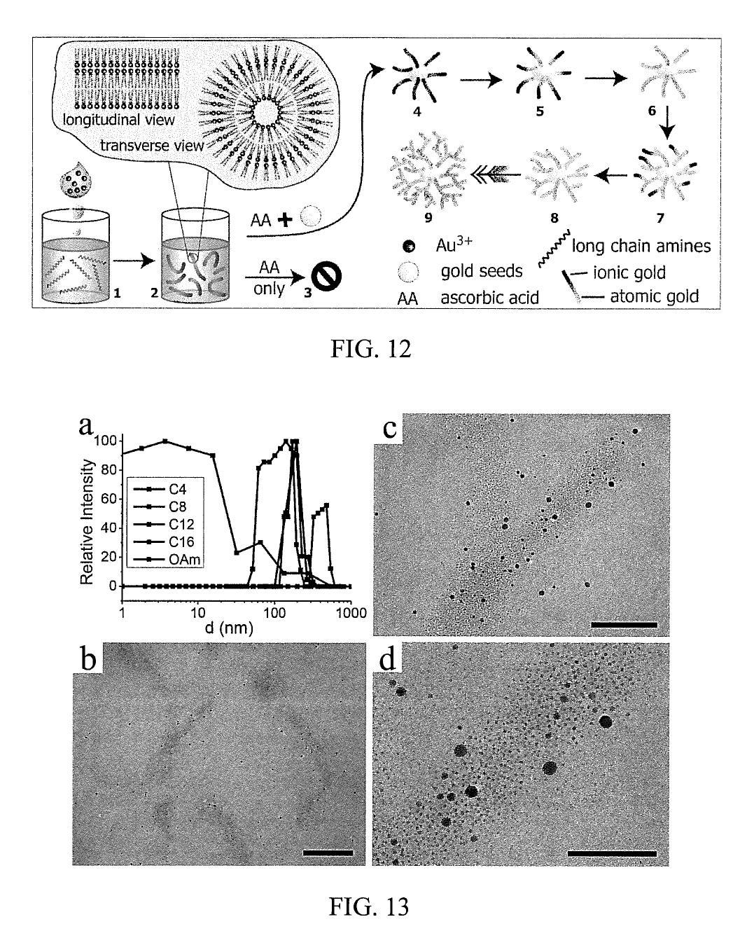

Investigations were made into the formation mechanism of the AuNDs. We discovered that the long chain amines could form rod-like nanostructures with HAuCl₄, which then prohibited the free diffusion of gold ions in the solution, due for example to their strong chelation with the amine group. Consequently, the confinement of gold ions inside the rod-like nanostructures would only allow the anisotropic deposition of atomic gold onto the seeds, which eventually ended up as a multiple-generation branched structure through a stepwise branch formation mechanism (FIGS. 12-14).

Previous reports of gold dendritic nanostructures have generally described particles in the range of tens of microns in overall size (J. X. Fang, X. N. Ma, H. H. Cai, X. P. Song, B. J. Ding, Nanoparticle-aggregated 3d monocrystalline gold dendritic nanostructures. Nanotechnology 17 (2006) 5841-5845; K. W. Hu, C. C. Huang, J. R. Hwu, W. C. Su, D. B. Shieh, C. S. Yeh, A new photothermal therapeutic agent: Core-free nanostructured AuAg₁₋ₓ dendrites. Chem.-Eur. J. 14 (2008) 2956-2964; Y. Qin, Y. Song, N. J. Sun, N. Zhao, M. X. Li, L. M. Qi, Ionic liquid-assisted growth of single-crystalline dendritic gold nanostructures with a three-fold symmetry. Chem. Mater. 20 (2008) 3965-3972; T. H. Lin, C. W. Lin, H. H. Liu, J. T. Sheu, W. H. Hung, Potential-controlled electrodeposition of gold dendrites in the presence of cysteine. Chem. Commun. 47 (2011) 2044-2046; M. Pan, H. Sun, J. W. Lim, S. R. Bakaul, Y. Zeng, S. X. Xing, et al., Seeded growth of two-dimensional dendritic gold nanostructures. Chem. Commun. 48 (2012) 1440-1442; J. S. Huang, X. Y. Han, D. W. Wang, D. Liu, T. Y. You, Facile synthesis of dendritic gold nanostructures with hyperbranched architectures and their electrocatalytic activity toward ethanol oxidation. ACS Appl. Mater. Interfaces 5 (2013) 9148-9154; J. Wunsche, L. Cardenas, F. Rosei, F. Cicoira, R. Gauvin, C. F. O. Graeff, et al., In situ formation of dendrites in eumelanin thin films between gold electrodes. Adv. Funct. Mater. 23 (2013) 5591-5598; Z.-Y. Lv, L.-P. Mei, W.-Y. Chen, J.-J. Feng, J.-Y. Chen, A.-J. Wang, Shaped-controlled electrosynthesis of gold nanodendrites for highly selective and sensitive sers detection of formaldehyde. Sensors and Actuators B: Chemical 201 (2014) 92-99). Gold particles of such size should not be confused with the nanoscale AuNDs of the present disclosure, as they have totally different properties and applications. Previously, colloidal AuNDs were prepared by using polymers (polypyrrole and polyaniline) and a non-commercial surfactant as the structural directing agent. However, these methods lack simplicity and manipulability, and none of them have control over the DB. In the conventional polymer-based methods, the as-prepared AuNDs are actually embedded in a polymer matrix which can hardly be removed due to its insolubility in most types of solvents (K. Huang, Y. J. Zhang, D. X. Han, Y. F. Shen, Z. J. Wang, J. H. Yuan, et al., One-step synthesis of 3d dendritic gold/polypyrrole nanocomposites via a self-assembly method. Nanotechnology 17 (2006) 283-288; M. Pan, S. X. Xing, T. Sun, W. W. Zhou, M. Sindoro, H. H. Teo, et al., 3d dendritic gold nanostructures: Seeded growth of a multi-generation fractal architecture. Chem. Commun. 46 (2010) 7112-7114). Thus, the surface of the NPs produced by such methods is not readily accessible for ligand exchange and molecular conjugation, making them unsuitable for biomedical applications. Further, the synthesis requires non-commercially available chemicals which makes the reproduction of AuNDs by other research groups a challenging task. In contrast, the AuNDs of the present disclosure are synthesized by commonly available long chain amines and the surface chemistry of the nanodendrite particles can be easily modified by different molecules. In this sense, the presently disclosed method produces AuNDs of a significantly better quality.

Manipulation of Degree of Branching

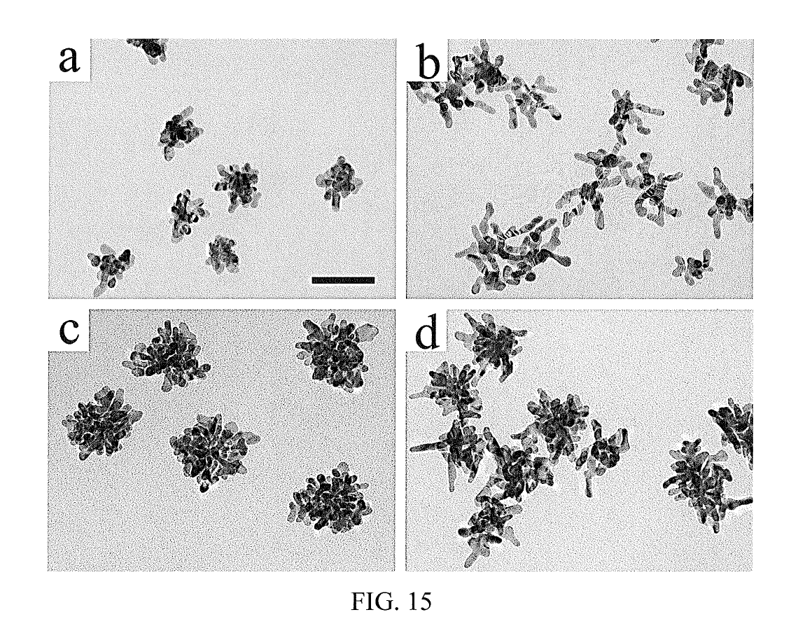

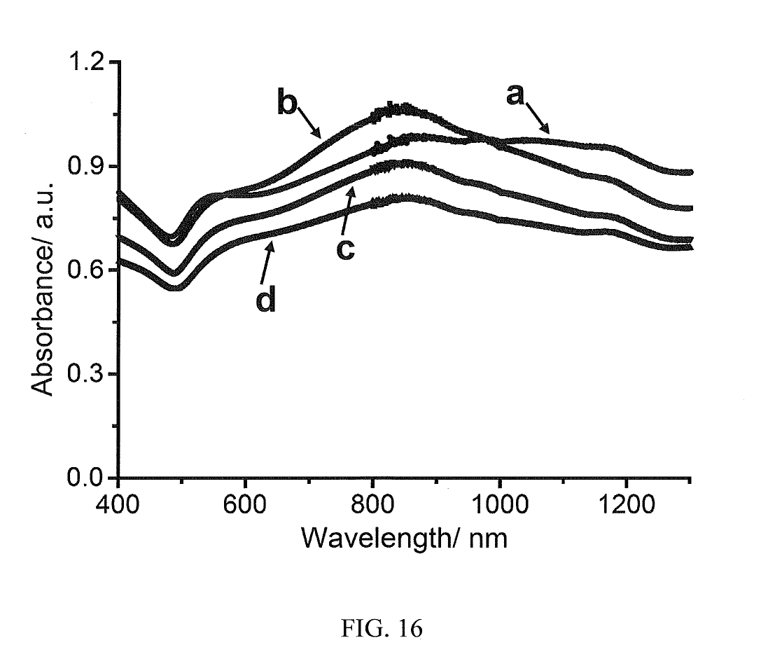

In previous reports of synthesizing dendritic NPs of Au—, Pt— and Pd-based metal and bimetals, no discernible difference in the DB of NPs was observed when adjusting the synthetic parameters, thus manipulation of DB was generally not achievable by the previous methods. However, in the methods of synthesis of the present disclosure, the DB of AuNDs can be controlled by (1) changing the solvent type in which the long chain primary amines are dissolved, and (2) by changing the concentrations of the long chain primary amines. For example, as shown in FIG. 15, when the synthesis is carried out in chloroform (b, d), instead of ethanol (a, c), the branch lengths in the resultant AuNDs are significantly longer, while the overall number of branches on a single nanodendrite particle (NP) is reduced. In other words, the AuNDs prepared in chloroform have a lower DB than those made in ethanol, which have a higher DB. In addition, the AuNDs made in chloroform also have much broader optical bands compared to those made in ethanol (FIG. 16).

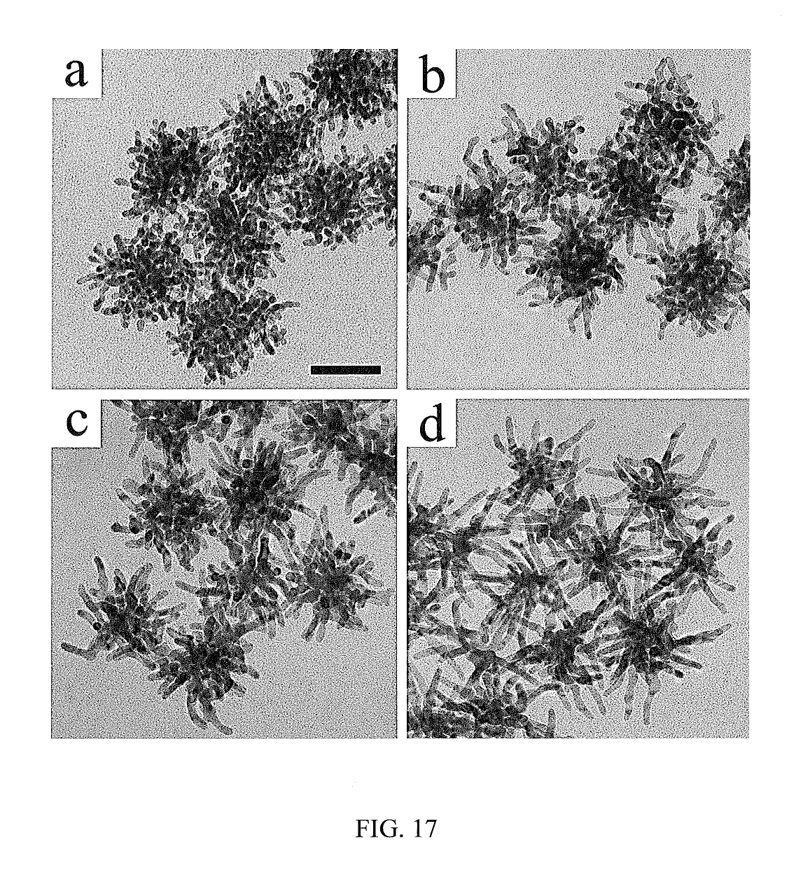

More sophisticated control over the DB can be achieved by manipulating the concentration of the long chain amine in the method. FIG. 17 shows AuNDs synthesized in 0.1, 0.4, 0.7 and 1.0 M of oleylamine. It can be seen that with increasing oleylamine concentration (a to d), the DB of AuNDs was reduced correspondingly while the lengths of individual branches increased. The fine manipulability of the presently disclosed synthetic method provides the ability to “tune” or adjust the DB and branch length on the AuNDs. This further enables, for the first time, the study of how the DB affects the optical and photothermal properties of dendritic NPs. For example, TEM images of three selected types of AuNDs for investigation with abruptly different DB are shown in FIG. 3.

Dependence of Optical and in Vitro Photothermal Properties of AuNDs on the DB

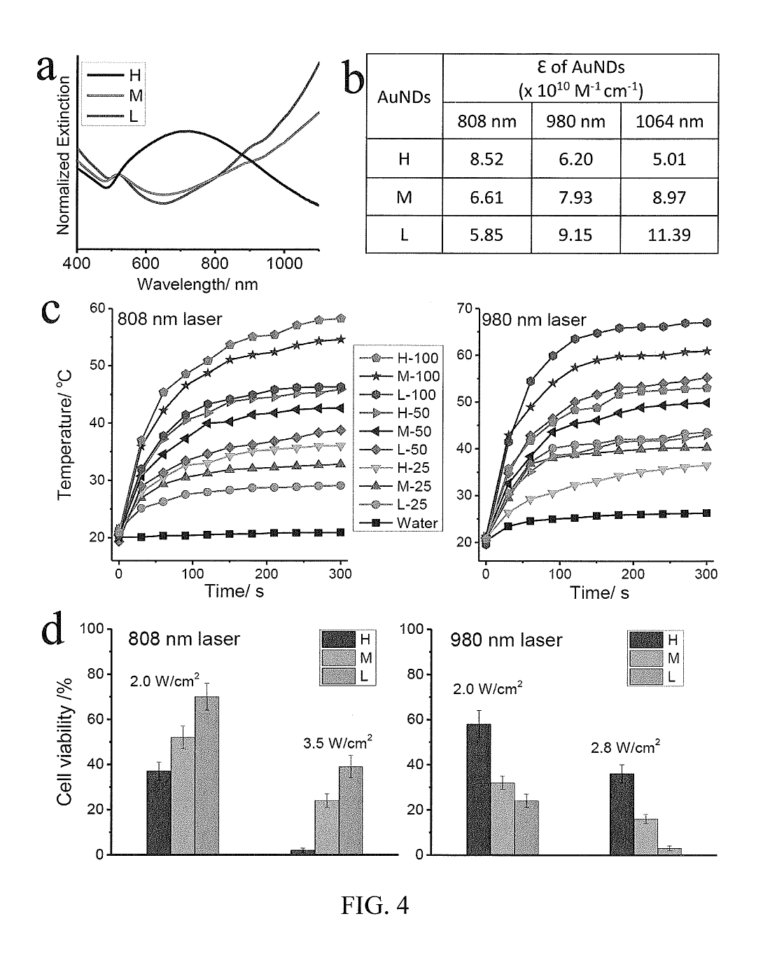

Metallic NPs with simple structures, such as nanospheres, nanorods, and nanocubes, usually show a sharp, narrow localized surface plasmon resonance (LSPR) band in their optical spectra. In contrast, the AuNDs are featured with complicated branched structures, as a result, their extinction bands are significantly broader. Here we refer to the AuNDs of high, medium and low DB as H-NDs, M-NDs and L-NDs, respectively. The H-NDs have an extinction band centered at around 700 nm; however M-NDs and L-NDs have relatively low peak intensity in this range, but tend to absorb more light beyond 1000 nm (FIG. 4a). The DB-dependent optical spectra of AuNDs should be attributed to their structural difference. Namely, NPs of high DB are mainly composed of short rod-like branches, which absorb intensively in the short wavelength region. This discovery is consistent with earlier reports that longer gold nanorods resulted in a shift of optical extinction to longer wavelength.

NPs that have strong extinction at the near infrared (NIR) region, within which the light can penetrate deep into tissues, are ideal candidates for photothermal applications. Most of the reported photothermal study was carried out by using NPs having absorption at 808, 980 and 1064 nm. The molar extinction coefficients (ε) of AuNDs of different DB at the above-mentioned three wavelengths are summarized in FIG. 4b. One can see that H-NDs have the highest ε at 808 nm, while M-NDs and L-NDs overide at both 980 and 1064 nm. We then carried out further study with 808 and 980 nm lasers to find out how such a difference in the optical properties would affect the performance of AuNDs in the photothermal application. We first collected the temperature profiles by directly irradiating aqueous solutions of AuNDs with lasers for 5 min and measuring the temperature change with an infrared camera. For all three different NPs concentrations (100, 50 and 25 μg/ml, respectively), the temperature profiles have shown a positive correlation to the ε of AuNDs. When irradiated with 808 nm laser, H-NDs always had a larger temperature increase, and with 980 nm laser, solutions containing L-NDs were hotter (FIG. 4c).

The photothermal efficiency of different AuNDs was further compared through in vitro study. In this study, the three types of AuNDs (100 μg/ml) were co-cultured with MCF-7 breast cancer cells for 12 h and then, after removing free NPs in the medium, the cells were treated by lasers for 5 min. The photothermal efficiency of NPs was then evaluated by cell viability, where lower viability represented better efficiency. The photothermal treatment for both types of lasers was initiated with a power density of 2.0 W/cm². Under this power density, AuNDs of high, medium and low DB resulted in a cell viability of 37, 52 and 70% by 808 nm laser and 58, 32 and 24% by 980 nm laser, respectively (FIG. 4d). A nearly 100% cell death was observed on H-NDs at 3.5 W/cm² (808 nm laser) and on L-NDs at 2.8 W/cm² (980 nm laser), while cells treated by the other two types of AuNDs in each group still had fairly high viability under the corresponding laser power density. These results show that the phototheiuial performance of the AuNDs is strongly dependent on their DB. NPs of high DB perform better with laser of shorter wavelength (808 nm); while those of lower DB are more suited for treatment at longer wavelength (980 and 1064 nm).

In Vivo Study of Photothermal Cancer Therapy with AuNDs of Different DB

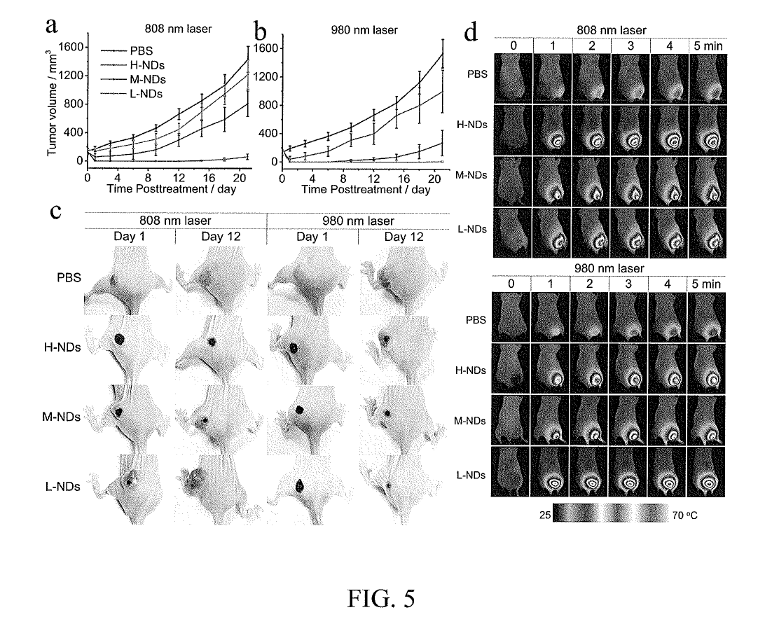

The in vitro photothermal study was extended to in vivo experiments. In this study, the AuNDs were introduced through intratumor injection. Photothermal therapy was applied with 5 min laser irradiation at a power density of 1.0 W/cm². Mice were housed for 3 weeks after the treatment, during which tumor volumes were recorded every 3 days. For photothermal therapy conducted by 808 nm laser, as shown in FIG. 5c, tumors injected by H-NDs were seen severely burnt and flattened on day 1 posttreatment, while the heat burn effect in the M-NDs and L-NDs groups was less evident. By day 12 posttreatment, the scarred area on the mice in the H-NDs group remained flat and been reduced notably. However, the tumors in the M-NDs and L-NDs groups became enlarged significantly, with the latter being the largest. In contrast, for groups using 980 nm laser as the light source (FIG. 5c), a reverse trend was observed. Tumors in all the three AuNDs groups were destructed heavily by the photothermal heat 1 day after the treatment, but by day 12 posttreatment, tumors in the L-NDs groups were almost gone, while tumors in the M-NDs and H-NDs groups became increasingly larger in volume. In addition, we also used an infrared camera to track the real-time temperature change in tumors during the photothermal treatment (FIG. 5d). The infrared images did reflect a positive correlation between the temperature increase inside tumors and the DBs under 808 nm laser irradiation and a negative correlation under 980 nm laser irradiation. This result is consistent with the tumor volume data shown in FIGS. 5a and b.

### EXAMPLE 3

Synthesis of dendritic structures by using triangular gold nanoplates and high-aspect-ratio gold nanorods as seeds

Synthesis of Nanoplates and Nanorods

Nanoplates and nanorods seeds were synthesized following methods know in the art. After the synthesis of the seeds, cetyltrimethylammonium bromide (CTAB) on the nanoparticles surface was replaced by PVP. The PVP replacement was done by suspending CTAB stabilized nanoparticles into a 1 wt % PVP aqueous solution, and stirring for 24 h. Afterwards, the nanoparticles were collected by centrifugation and suspended stably into ethanol.

Synthesis of Dendritic Structure onto Nanoplates and Nanorods

The synthesis was performed in 4 ml of 0.1 M hexadecylamine ethanolic solution. The amount of seeds was kept constant; variation was made to the addition of HAuCl₄, and correspondingly the reductant, ascorbic acid. The amounts of HAuCl₄ (60 mM) and ascorbic acid (0.4 M) used for three differently overgrown dendritic structures in FIG. 3 were 3 μl/3 μl, 6 μl/5 μl, and 18 μl/10 μl, respectively. After the synthesis, a same phase transfer process was also applied to these nanoparticles before TEM samples were made.

Catalytic study of AuNDs

Synthesis of 4, 7, and 10 nm Spherical AuNPs

4 nm AuNPs were synthesized by NaBH₄ reduction of HAuCl₄ in the presence of sodium citrate. Briefly, 500 μl of 10 mM aqueous HAuCl4 was first diluted with 18 ml of water, followed by addition of 125 μl of 40 mM sodium citrate. Then immediately 500 μl of freshly prepared 0.1 M NaBH₄ was injected under vigorous stirring to obtain the 4 nm AuNPs. Afterwards, 100 mg of PVP 10 K was added into the NPs solution and stirred for 30 min to achieve the PVP coating of the surface.

7 nm and 10 nm AuNPs were prepared through the seed-mediated process. Into the solution containing 3 ml of water, 120 μl of 10 mM HAuCl₄, and 20 mg of PVP 10 K, 1 ml of the above prepared 4 nm AuNP solution was added, then 100 μl of 80 mM aqueous ascorbic acid was added dropwise to obtain the 7 nm AuNPs. The preparation of 10 nm AuNPs was carried out in the same way except that the seed solution used was 1 ml of the 7 nm AuNPs.

Formation mechanism of the AuNDs

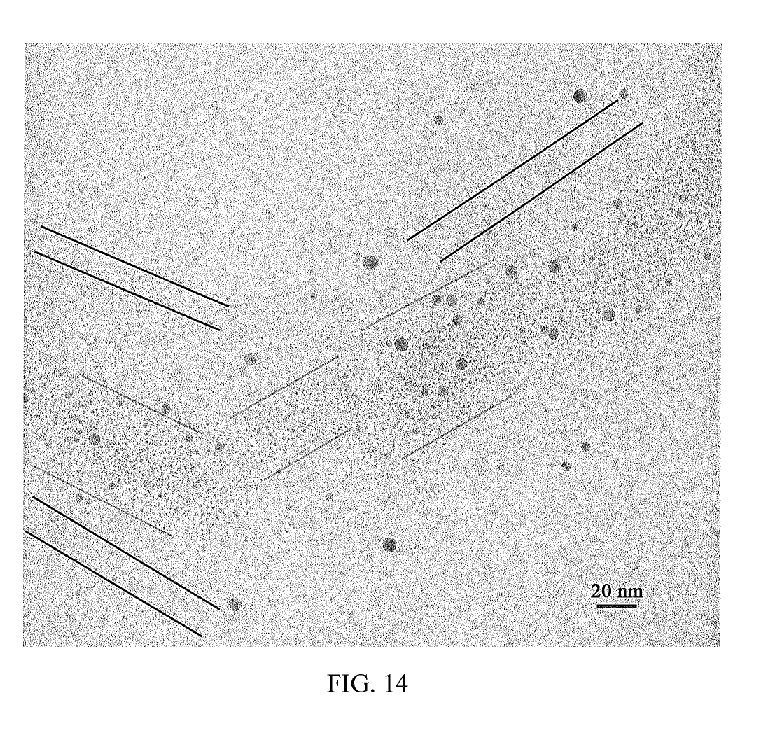

Investigations into the AuND formation mechanism began with the ethanolic solutions of the amines before and after the addition of HAuCl₄ by Dynamic Light Scattering (DLS). The DLS has been used as an effective tool to analyze molecular assembly behavior in solution phase. In all of the pure primary amine solutions (CₙH₂ₙ₊₁NH₂, n=4, 8, 12, 16 and oleylamine), the DLS measurement showed an average particle size of zero, suggesting that the amines do not self-assemble in ethanol under the experimental conditions. After the addition of HAuCl₄, however, particles of 50-400 nm were observed immediately in the dodecylamine, hexadecylamine and oleylamine solutions; meanwhile more than 90% particles in the octylamine solution were less than 15 nm and no particles were formed in the butylamine solution (FIG. 13a). The particles detected by DLS can be collected by centrifugation. We then employed TEM to study the detailed structure of the particles. In our initial TEM examination, we noticed some rod-like shadows, which can hardly be pictured. The TEM grid with the particles deposited was then heated under 60° C. for 12 h, during which Au³⁺ was reduced slowly by the amines. As shown in FIG. 13b, the rod-like shape was retained successfully after the heat treatment. Under higher magnification TEM, it can be discerned clearly that numerous tiny gold nanoparticles are embedded inside the rod-like profile (FIGS. 13c-d). In addition, the diameter of the rod-like structures shown in FIG. 13 is about 40-50 nm, however, we believe that these thick rods are composed of bundles of thin rods that might have been assembled together during the treatment for TEM observation (FIG. 14).

The DLS and TEM studies collectively indicate that long chain amines tend to self-assemble into rod-like nanostructures upon the addition of HAuCl₄. Hence, without wishing to be bound by theory, a possible mechanism of the dendrite formation is proposed (FIG. 12). Although the detailed arrangement of molecules inside the rods is not quite clear, we hypothesize that it is most likely a multiple-layered stacking (FIG. 12, steps 1-2), commonly seen for amphiphilic molecules such as the long chain amines. Due to the strong chelation with amine groups, gold ions are confined inside the rod-like nanostructures, and therefore, their free diffusion in the solution is prohibited. Such chelation interaction also makes the ascorbic acid unable to effectively reduce Au³⁺ to Au⁰ alone (FIG. 12, step 3). With pre-synthesized seeds as a catalyst, the reduction reaction can be initiated once the rod-like nanostructures are in contact with the seeds. Because of the confinement of Au³⁺, deposition of gold atoms can only take place anisotropically along the longitudinal direction of the rod, which will end up as first generation branches grown on the surface of seeds (FIG. 12, steps 4-6). The first generation branches can be employed immediately as a catalyst for the deposition of second generation branches, and the growing process will continue in such a manner to eventually form a multi-generation branched structure (FIG. 12, steps 7-9). In addition, the softness of the rods formed by Au³⁺ and long chain amines might also have contributed to the branch formation.

### EXAMPLE 4

General Synthetic Protocols for AuNDs

In certain embodiments, to perform a synthesis, 4 ml of 0.1 M ethanolic solution of amines (butylamine, octylamine, dodecylamine, hexadecylamine, octadecylamine, oleylamine), 20 μl of 60 mM HAuCl₄ in ethanol, varying amount of seeds, and 15 μl of 0.4 M ascorbic acid in methanol were added subsequently into the reaction vial. The solutions were then mixed vigorously by hand for a few seconds, and then left on a rocker shaker for about 30 min, during which AuNDs were formed. The nanoparticles were then centrifuged, washed once with chloroform and ethanol and finally redispersed into 2 ml of THF.

In accordance with the present disclosure, AuNDs can be formed using various combinations of synthetic parameters, such as HAuCl₄ concentration, ascorbic acid concentration, and seed concentration, and not necessarily the amounts indicated herein. Further, AuNDs can also be obtained using other solvents, such as water, alcohols (including but not limited to methanol, ethanol, isopropanol, and alcohols with longer carbon chain), as well as other, non-polar, organic solvents, in which the long chain amines can dissolve well to form a homogeneous solution, such as but not limited to chloromethane, dichloromethane, and chloroform. In addition to the amines mentioned above, other types of long chain amines, with saturated and monounsaturated and polyunsaturated carbon chains, can also lead to the formation of AuNDs.

Controlling Degree of Branching of AuNDs

As explained elsewhere herein, the DB of AuNDs can be adjusted or “tuned” by using alternate solvent types and concentrations of long chain amines. In general, highly-branched AuNDs with short branch lengths are usually obtained using amines dissolved in polar solvents, (e.g., alcohols such as ethanol) and lesser-branched AuNDs with longer branch lengths are made in less polar or “nonpolar” solvents, such as chloroform. Further, use of amine solutions with higher amine concentrations tend to generate AuNDs with longer branch length. The following include examples of how to make AuNDs of high, medium and low degree of branching. The recipes in each group generates AuNDs of varying degrees of branching. Some of the recipes among different groups are the same. All the AuNDs generated by these recipes have different optical absorption bands toward the solar spectra, some absorb more in the visible range, some absorb more in the near infrared range, and some evenly cover the entire visible and near infrared range of the solar spectrum, enabling case-by-case selection of a specific type of AuNDs for certain applications.

Group 1

1. High degree of branching: 4 ml of 0.1 M hexadecylamine in ethanol +20 μl of 60 mM HAuCl₄ in ethanol+seed of varying amount +15 μl of 0.4 M ascorbic acid.

2. Medium degree of branching: 4 ml of 0.7 M oleylamine in chloroform +20 μl of 60 mM HAuCl₄ in ethanol+seed of varying amount +15 μl of 0.4 M ascorbic acid.

3. Low degree of branching: 4 ml of 1.0 M oleylamine in chloroform +20 μl of 60 mM HAuCl₄ in ethanol+seed of varying amount +15 μl of 0.4 M ascorbic acid.

Group II

1. High degree of branching: 4 ml of 0.1 M hexadecylamine in ethanol +20 μl of 60 mM HAuCl₄ in ethanol+seed of varying amount +15 μl of 0.4 M ascorbic acid;

2. Low degree of branching: 4 ml of 0.1 M hexadecylamine in chloroform +20 μl of 60 mM HAuCl₄ in ethanol+seed of varying amount +15 μl of 0.4 M ascorbic acid;

Group III

1. High degree of branching: 4 ml of 0.1 M oleylamine in chloroform +20 μl of 60 mM HAuCl₄ in ethanol+seed of varying amount +15 μl of 0.4 M ascorbic acid;

2. Low degree of branching: 4 ml of 1.0 M oleylamine in chloroform +20 μl of 60 mM HAuCl₄ in ethanol+seed of varying amount +15 μl of 0.4 M ascorbic acid;

For oleylamine in between 0.1-1.0 M range, degree of branching of the AuNDs decreases slowly with increasing concentration of oleylamines.

The results provided above show for the first time that DB of NDs in general and AuNDs in particular can be manipulated by using long chain primary amines as the structural directing agents. This work also represents a novel and simple method to prepare high quality AuNDs based on commonly available chemicals. This discovery enabled us to investigate how the DB would impact the various properties of AuNDs. We found that the manipulable DB brought tunable optical properties of AuNDs in a wide near infrared range, which further allowed us to discover, through both in vitro and in vivo experiments, the wavelength-dependent photothermal properties of AuNDs.

In at least a certain embodiment, the present disclosure is directed to a method of forming gold dendritic nanoparticles, comprising in no particular order: (1) providing a quantity of seed nanoparticles comprising elemental gold, (2) providing an amine-solvent solution comprising a long chain primary amine disposed in a solvent, (3) providing an ionic gold solution, (4) providing a reducing reagent solution; then combining the quantity of seed nanoparticles, the amine-solvent solution, the ionic gold solution, and the reducing reagent solution in a container to form a mixture; agitating the mixture for a duration of time sufficient to cause formation of dendritic gold nanoparticles in the mixture; and isolating the dendritic gold nanoparticles from the mixture.

The ionic gold solution may comprise HAuCl₄. The solvent of the amine-solvent solution may comprise an alcohol. The alcohol may be selected from the group consisting of methanol, ethanol, propanol, isopropanol, and butanol, and combinations thereof. The solvent of the amine-solvent solution may comprise an organic solvent. The organic, non-polar solvent may be selected from the group consisting of chloroform, chloromethane, dichloromethane, and diethyl ether, and combinations thereof. The solvent of the amine-solvent solution may comprise a solvent mixture comprising an alcohol and an organic solvent. The solvent of the solvent mixture may comprise ethanol and chloroform. The amine of the amine-solvent solution may be selected from the group consisting of butylamine, octylamine, dodecylamine, hexadecylamine, octadecylamine, and oleylamine, and combinations thereof. The amine of the amine-solvent solution may have a carbon chain length selected from the group consisting of 4, 5, 6, 7, 8, 9, 10, 11, 12, 13, 14, 15, 16, 17, 18, 19, 20, 21, 22, 23, 24, 25, 26, 27, 28, 29, and 30, and amine combinations thereof. That is, the amine-solvent solution may comprise a plurality of amines of different chain length, such as both octadecylamine, and oleylamine. The amines of the amine-solvent solution may comprise both saturated carbon chains and unsaturated carbon chains.

The reducing reagent solution may comprise at least one of ascorbic acid, citric acid, ascorbate ions, hydrazine, and hydroxylamine. The seed nanoparticles may comprise a coating selected from the group consisting of polyvinylpyrrolidone (PVP), polyvinyl alcohol (PVA), polyethylene glycol (PEG), and combinations thereof. The dendritic gold nanoparticles may have diameters in a size range of about 20 nm to about 1000 nm. The dendritic gold nanoparticles may absorb light in a wavelength range of, for example, about 300 nm to about 2500 nm, a range of about 400 nm to about 1600 nm, a range of about 750 nm to about 2500 nm, a range of about 750 nm to about 1600 nm, or a range of about 800 nm to about 2000 nm. The dendritic gold nanoparticles may be formed into a continuous film having a thickness in a range of about 20 nm to about 100 μm.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims. Applicants reserve the right to amend, add to, or replace the claims indicated herein below in subsequent patent applications.

What is claimed is:

1. A method of forming gold dendritic nanoparticles, comprising:
providing a quantity of seed nanoparticles comprising elemental gold;
providing an amine-solvent solution comprising at least one long chain primary amine disposed in a solvent;
providing an ionic gold solution;
providing a reducing reagent solution;
combining the quantity of seed nanoparticles, the amine-solvent solution, the ionic gold solution, and the reducing reagent solution in a container to form a mixture, and agitating the mixture for a duration of time sufficient to cause formation of dendritic gold nanoparticles in the mixture; and
isolating the dendritic gold nanoparticles from the mixture.

2. The method of claim 1, wherein the ionic gold solution comprises HAuCl₄.

3. The method of claim 1, wherein the solvent of the amine-solvent solution comprises an alcohol.

4. The method of claim 3, wherein the alcohol is selected from the group consisting of methanol, ethanol, propanol, isopropanol, and butanol, and combinations thereof.

5. The method of claim 1, wherein the solvent of the amine-solvent solution comprises an organic solvent.

6. The method of claim 5, wherein the organic, non-polar solvent is selected from the group consisting of chloroform, chloromethane, dichloromethane, and diethyl ether, and combinations thereof.

7. The method of claim 1, wherein the solvent of the amine-solvent solution comprises a solvent mixture comprising an alcohol and an organic solvent.

8. The method of claim 7, wherein the solvent of the solvent mixture comprises ethanol and chloroform.

9. The method of claim 1, wherein the at least one long chain primary amine of the amine-solvent solution is selected from the group consisting of butylamine, octylamine, dodecylamine, hexadecylamine, octadecylamine, and oleylamine, and combinations thereof.

10. The method of claim 1, wherein the at least one long chain primary amine of the amine-solvent solution has a carbon chain length selected from the group consisting of 4, 5, 6, 7, 8, 9, 10, 11, 12, 13, 14, 15, 16, 17, 18, 19, 20, 21, 22, 23, 24, 25, 26, 27, 28, 29, and 30, and amine combinations thereof.

11. The method of claim 1, wherein the at least one long chain primary amine of the amine-solvent solution comprises a saturated carbon chain.

12. The method of claim 1, wherein the at least one long chain primary amine of the amine-solvent solution comprises an unsaturated carbon chain.

13. The method of claim 1, wherein the reducing reagent solution comprises at least one of ascorbic acid, citric acid, ascorbate ions, hydrazine, and hydroxylamine.

14. The method of claim 1, wherein the seed nanoparticles comprise a coating selected from the group consisting of polyvinylpyrrolidone (PVP), polyvinyl alcohol (PVA), polyethylene glycol (PEG), and combinations thereof.

15. The method of claim 1, wherein the dendritic gold nanoparticles have diameters in a size range of about 20 nm to about 1000 nm.

16. The method of claim 1, wherein the dendritic gold nanoparticles absorb light in a wavelength range of about 300 nm to about 2500 nm.

17. The method of claim 1, wherein the dendritic gold nanoparticles absorb light in a wavelength range of about 400 nm to about 2000 nm.

18. The method of claim 1, wherein the dendritic gold nanoparticles absorb light in a wavelength range of about 750 nm to about 2000 nm.

19. The method of claim 1, wherein the dendritic gold nanoparticles absorb light in a wavelength range of about 750 nm to about 1600 nm.

20. The method of claim 1, comprising forming the dendritic gold nanoparticles into a continuous film having a thickness in a range of about 20 nm to about 100 μm.
